(19) United States
(12) Patent Application Publication
(10) Pub. No.: US 2022/0351000 A1
(43) Pub. Date: Nov. 3, 2022

(54) METHOD AND APPARATUS FOR CLASSIFYING NODULES IN MEDICAL IMAGE DATA

(71) Applicant: Cygnus-AI Inc., Williston, FL (US)

(72) Inventors: Scott Anderson MIDDLEBROOKS, Beaverton, OR (US); Adrianus Cornelis KOOPMAN, BM Hilversum (NL); Ari David GOLDBERG, Chicago, IL (US); Henricus Wilhelm VAN DER HEIJDEN, Turnhout (BE)

(73) Assignee: Cygnus-AI Inc., Williston, FL (US)

(21) Appl. No.: 17/306,085

(22) Filed: May 3, 2021

(51) Int. Cl. G06K 9/62; G06K 9/46; G16H 50/20; G16H 30/20

(52) U.S. Cl. CPC G06K 9/628; G06K 9/4647; G06K 9/6277; G16H 50/20; G16H 30/20; G06K 2209/053; G06K 9/6256; G06K 9/6262

(57) ABSTRACT

Disclosed are methods and systems for processing medical image data. The method comprising inputting, with one or more processors of one or more computation devices, medical image data into a model for nodule detection; calculating, for at least one nodule detected by the model for nodule detection, a nodule histogram of all voxel intensities of said nodule; determining, from each nodule histogram, a nodule classification among a plurality of nodule classifications for the at least one nodule.

### FIELD OF THE DISCLOSURE

The disclosure relates to computer-aided diagnosis (CAD). The disclosure also relates to a method and a platform or system for using machine learning algorithms for processing medical data. In particular, the disclosure relates to a method and apparatus for classifying nodules in medical image data.

### BACKGROUND OF THE DISCLOSURE

Advances in computed tomography (CT) allow early detection of cancer, in particular lung cancer which is one of the most common cancers. As a result, there is increased focus on using regular low-dose CT screenings to ensure early detection of the disease with improved chances of success of the following treatment. This increased focus leads to an increased workload for professionals such as radiologists who have to analyze the CT screenings.

To cope with the increased workload, computer-aided detection (CADe) and computer-aided diagnosis (CADx) systems are being developed. Hereafter both types of systems will be referred to as CAD systems. CAD systems can detect lesions (e.g. nodules) and subsequently classify them as malignant or benign. A classification need not be binary, it can also include a stage of the cancer. Usually, a classification is accompanied with a confidence value as calculated by the CAD system.

Hereafter the term “model” will be used to indicate a computational framework for performing one or more of a segmentation and a classification of imaging data. The segmentation, identification of regions of interest, and/or the classification may involve the use of a machine learning (ML) algorithm. The model comprises at least one decision function, which may be based on a machine learning algorithm, which projects the input to an output. Where the term machine learning is used, this also includes further developments such as deep (machine) learning and hierarchical learning.

Whichever type of model is used, suitable training data needs to be available to train the model. In addition, there is a need to obtain a confidence value to be able to tell how reliable a model outcome is. Most models will always give a classification, but depending on the quality of the model and the training set, the confidence of the classification may vary. It is of importance to be able to tell whether or not a classification is reliable.

While CT was used as an example in this introduction, the disclosure can also be applied to other modalities, such as ultrasound, Magnetic Resonance Imaging (MRI), Positron Emission Spectrograph (PET), Single Photon Emission Computed Tomography (SPECT), X-Ray, and the like.

### SUMMARY OF THE DISCLOSURE

It is an object of this disclosure to provide a method and apparatus for classifying nodules in imaging data.

Accordingly, the disclosed subject matter provides a method for processing medical image data, the method comprising:
inputting, with one or more processors of one or more computation devices, medical image data into a model for nodule detection;
calculating, for at least one nodule detected by the model for nodule detection, a nodule histogram of all voxel intensities of said nodule;
determining, from each nodule histogram, a nodule classification among a plurality of nodule classifications for at least one nodule.

Further embodiments are disclosed in attached dependent claims 2-8.

The disclosure further provides a computer system comprising one or more computation devices in a cloud computing environment and one or more storage devices accessible by the one or more computation devices, wherein the one or more computing devices comprise one or more processors, and wherein the one or more processors are programmed to:
inputting medical image data into a model for nodule detection;
calculating, for at least one nodule detected by the model for nodule detection, a nodule histogram of all voxel intensities of said nodule;
determining, from each nodule histogram, a nodule classification among a plurality of nodule classifications for at least one nodule.

Further embodiments are disclosed in attached dependent claims 10-18.

The disclosure further provides a computer program product comprising instructions which, when executed on a processor, cause said processor to implement one of the methods or systems as described above.

### BRIEF DESCRIPTION OF THE FIGURES

Embodiments of the present disclosure will be described hereinafter, by way of example only, with reference to the accompanying drawings which are schematic in nature and therefore not necessarily drawn to scale. Furthermore, like reference signs in the drawings relate to like elements.

FIG. 1 schematically shows an overview of a workflow according to embodiments of the disclosed subject matter;

FIGS. 2a and 2b schematically show a method of classifying nodules according to an embodiment of the disclosed subject matter;

FIG. 3 schematically shows a model for nodule detection according to an embodiment of the disclosed subject matter;

FIG. 4 schematically shows a nodule histogram of all voxel intensities of a nodule according to embodiments of the disclosed subject matter;

FIG. 5 schematically shows a method for determining a nodule classification according to an embodiment of the disclosed subject matter,

FIG. 6 schematically shows a further method of classifying nodules according to an embodiment of the disclosed subject matter; and

FIG. 7 schematically shows an encoder-decoder pair according to an embodiment of the disclosed subject matter.

### DETAILED DESCRIPTION

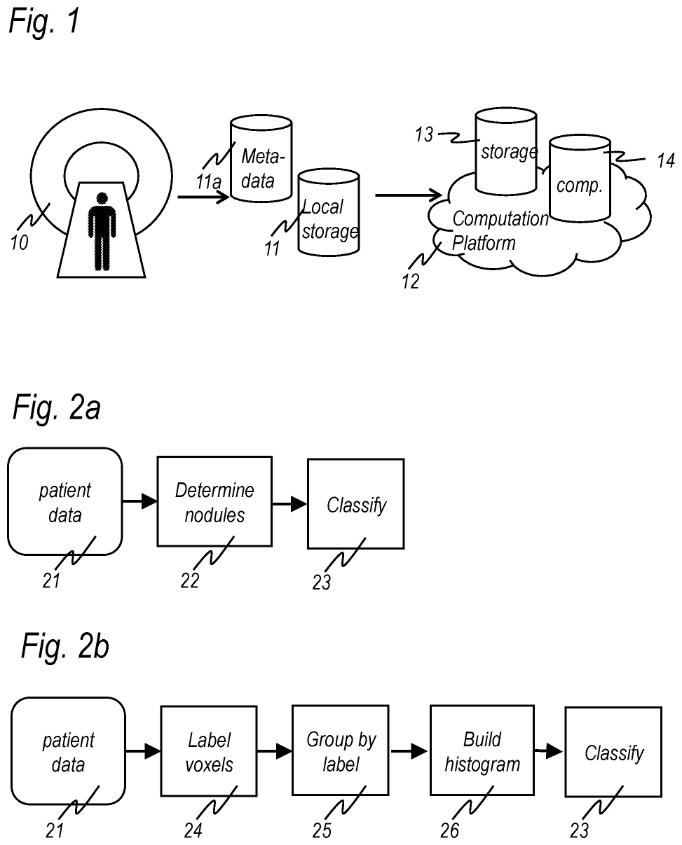

FIG. 1 schematically shows an overview of a workflow according to embodiments of the disclosed subject matter. A patient is scanned in scanning device 10. The scanning device 10 can be any type of device for generating diagnostic image data, for example an X-Ray device, a Magnetic Resonance Imaging (MRI) scanner, PET scanner, SPECT device, or any general Computed Tomography (CT) device. Of particular interest are low-dose X-Ray devices for regular and routine scans. The various types of scans can be further characterized by the use of a contrast agent, if any. The image data is typically three-dimensional (3D) data in a grid of intensity values, for example 512×512×256 intensity values in a rectangular grid.

In the following, the example of a CT device, in particular a CT device for low dose screenings, will be used. However, this is only exemplary. Aspects of the disclosure can be applied to any instantiation of imaging modality, provided that it is capable of providing imaging data. A distinct type of scan (X-Ray CT, low-dose X-Ray CT, CT with contrast agent X) can be defined as a modality.

The images generated by the CT device 10 (hereafter: imaging data) are sent to a storage 11 (step S1). The storage 11 can be a local storage, for example close to or part of the CT device 10. It can also be part of the IT infrastructure of the institute that hosts the CT device 10. The storage 11 is convenient but not essential. The data could also be sent directly from the CT device 10 to computation platform 12. The storage 11 can be a part of a Picture Archiving and Communication System (PACS).

All or parts of the imaging data is then sent to the computation platform 12 in step S2. In general it is most useful to send all acquired data, so that the computer models of platform 12 can use all available information. However, partial data may be sent to save bandwidth, to remove redundant data, or because of limitations on what is allowed to be sent (e.g. because of patient privacy considerations). The data sent to the computation platform 12 may be provided with metadata from scanner 10, storage 11, or further database 11a. Metadata can include additional data related to the imaging data. For example statistical data of the patient (gender, age, medical history) or data concerning the equipment used (type and brand of equipment, scanning settings, etc).

Computation platform 12 comprises one or more storage devices 13 and one or more computation devices 14, along with the necessary network infrastructure to interconnect the devices 13, 14 and to connect them with the outside world, preferably via the Internet. It should be noted that the term “computation platform” is used to indicate a convenient implementation means (e.g. via available cloud computing resources). However, embodiments of the disclosure may use a “private platform”, i.e. storage and computing devices on a restricted network, for example the local network of an institution or hospital. The term “computation platform” as used in this application does not preclude embodiments of such private implementations, nor does it exclude embodiments of centralized or distributed (cloud) computing platforms. The computation platform, or at least elements 13 and/or 14 thereof, can be part of a PACS or can be interconnected to a PACS for information exchange, in particular of medical image data.

The imaging data is stored in the storage 13. The central computing devices 14 can process the imaging data to generate feature data as input for the models. The computing devices 14 can segment imaging data. The computing devices 14 can also use the models to classify the (segmented) imaging data. More functionality of the computing devices 14 will be described in reference to the other figures.

A work station (not shown) for use by a professional, for example a radiologist, is connected to the computation platform 12. Hereafter, the terms “professional” and “user” will be used interchangeably. The work station is configured to receive data and model calculations from the computation platform. The work station can visualize received raw data and model results.

FIG. 2a schematically shows a method of classifying nodules according to an embodiment of the disclosed subject matter.

Medical image data 21 is provided to the model for nodule detection. The medical image data 21 can be 3D image data, for example a set of voxel intensities organized in a 3D grid. The medical image data can be organized into a set of slices, where each slice includes intensities on a 2D grid (say, an x-y grid) and each slice corresponds to a position along a z-axis as 3^{rd} dimension. The data can for example be CT or MRI data. The data can have a resolution of for example 512×512×512 voxels or points.

The model for nodule detection, used in action 22 to determine nodules from the medical image data 21, may be a general deep learning model or machine learning model, in particular a deep neural network, such as a Convolutional Neural Network (CNN or ConvNet), a U-net, a Residual Neural Network (RNN or Resnet), or a Transformer deep learning model. The model can comprise a combination of said example models. The model can be trained in order to detect nodules or lesions. The model may comprise separate segmenting and classification stages, or alternatively it may segment and classify each voxel in one pass. The output of the model is a set of one or more detected nodules (assuming there is at least one or more nodules in the input data).

Finally, in action 23, the nodule's quality is classified based on the histogram. Further details are provided in reference to FIG. 5. The classification may be one of ground glass (also called non-solid), part solid, solid, and calcified. Based on the classification, and segmented size estimation, a lung-RADS score may be determined or at least estimated. Lung-RADS comprises a set of definitions designed to standardize lung cancer screening CT reporting and management recommendations, developed by the American College of Radiology.

FIG. 2b schematically shows a further method of classifying nodules according to an embodiment of the disclosed subject matter, in which the function of the model for nodule detection 22 is further detailed in actions 24 and 25. The other parts of this embodiment are as the embodiment of FIG. 2a and will not be repeated here.

In action 24, all or almost all voxels in the data set (possibly excluding voxels near the boundaries of the 3D grid) are processed by the model for label prediction. The predicted label is selected from a set of labels that at least includes one “nodule” label and at least one “non-nodule” label. It should be noted that said model for nodule classification may be capable of determining other characteristics of a voxel besides whether or not said voxel is part of a nodule or not. Such a model may for example also predict voxels as corresponding to bone or tissue.

After action 24, all or nearly all voxels in the medical image data 21 have been predicted as nodule or something other than nodule. The voxels predicted as nodule are grouped together in action 25. Grouping may be done using connected component labelling or using another grouping algorithm known to the skilled person. As a result of the grouping, each group represents one nodule.

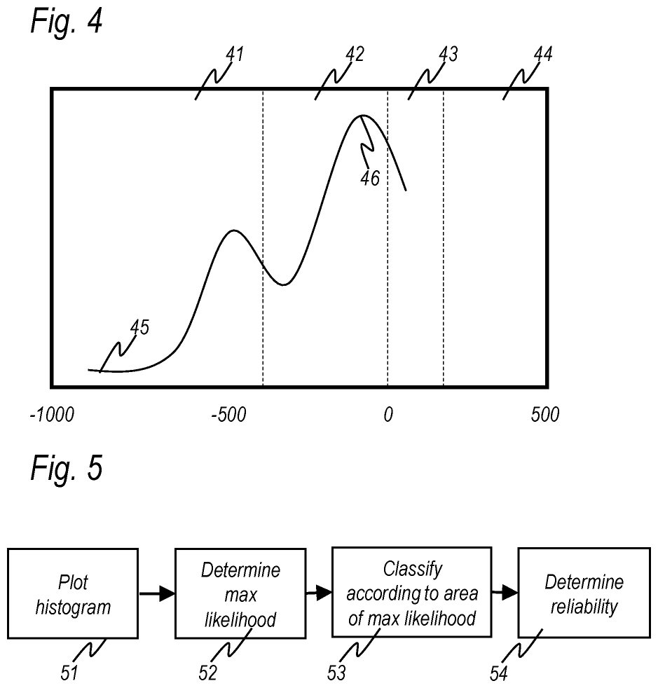

In action 26, for each detected nodule, a respective histogram is created based on the intensities of all data voxels that are part of the nodule (so, part of the nodule's group). More details are provided in reference to FIG. 4. Finally, in step 23 the nodule is classified based on the histogram.

Applicant has found that the procedure according FIG. 2b, where first all voxels are labelled and then grouped, works particularly well with the histogram based nodule classification that will be described herein below.

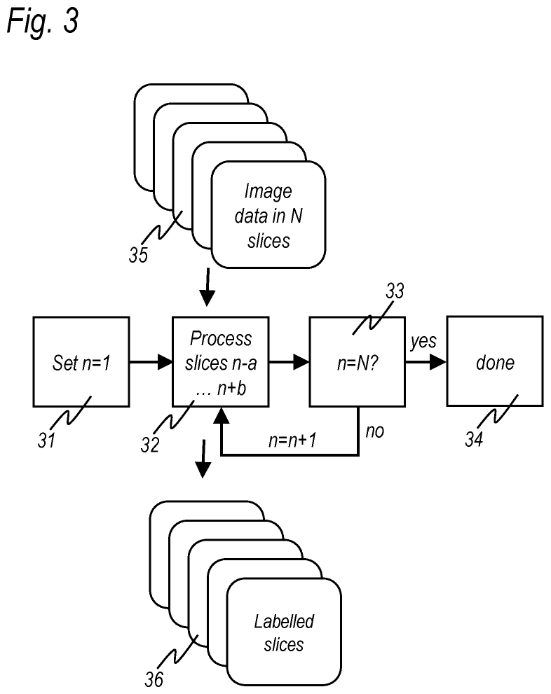

FIG. 3 schematically shows a model for nodule detection according to an embodiment of the disclosed subject matter. It is an example of how action 26 can be implemented advantageously.

The model involves an iteration over a set of N 2D image slices that together form 3D image data 35. The algorithm starts at slice n=1 (action 31) and repeats with increasing n until n=N (action 33, 34). In every iteration (action 32), a context of a+b slices n−a to n+b is evaluated. In a symmetrical processing method, a=b, so that the evaluated slice is in the middle of the data set. This is, however, not essential. Near the boundaries of the data set (n≤a or n≥b), special measures must be taken. These slices can be skipped, or data “over the boundary” can be estimated, e.g. by extrapolation or repetition of the boundary values.

As mentioned before, the prediction of the slice of data in action 32 can be done using a CNN or another machine learning model. The output is a predicted slice, where each voxel in the slice (again, possibly excluding boundary voxels) has a nodule or non-nodule label, and associated classification probability. After the full set of input slices 35 is processed, a labelled set of output slices 36 is obtained.

The output slices 36 can be provided to the grouping method in action 27 of FIG. 2b.

FIG. 4 schematically shows a nodule histogram of all voxel intensities of a nodule according to embodiments of the disclosed subject matter. The horizontal axis represents voxel intensity in an appropriate unit. In the example of FIG. 4, Hounsfield units (HU) are used, but other units may be used as well. The horizontal axis is divided into intensity bins (not shown), while the vertical axis is proportional to the number of voxels in an intensity bin.

The horizontal range is divided into a number (in the present example, four) intensity ranges 41, 42, 43, 44. Intensity range 41 represents ground glass (also called non-solid), intensity range 42 represents part solid, intensity range 43 represents solid, and intensity range 44 represents calcified. The intensity ranges can be fixed or determined dynamically by a model or algorithm. There can also be any number of intensity ranges, depending on the number classifications.

Curve 45 represents an example histogram for a nodule. The example histogram has one local maximum and a global maximum 46. In general, the histogram may not have a local maximum. The intensity where the histogram has a global maximum 46 is considered the maximum likelihood intensity.

FIG. 5 schematically shows a method for determining a nodule classification according to an embodiment of the disclosed subject matter. In action 51, the nodule histogram 45 is determined as described in reference to FIG. 4. In action 52, the intensity of maximum likelihood is determined, which is the intensity corresponding to the global maximum 46 in the histogram 45. The range 41, 42, 43, 44 in which the maximum likelihood intensity is located is determined in action 53. In the example of FIG. 4, the maximum 46 is in region 42, corresponding to the part solid classification.

In optional step 54 a reliability of the determined classification is made. For example, this determination can be based on one or more distances of the maximum likelihood intensity to an intensity range boundary and the difference between the maximum value and the highest local maximum (if any). The determination can include information on which other classification is closest. E.g. in the example of FIG. 4, the classification is “part solid” otherwise “solid”, because maximum 46 is in range 42 (part solid) but relatively close to range 43 (solid). The predicted probabilities may be taken into account, by using a probability weighted histogram, or discounting probability values below a certain threshold, or by taking the distribution of prediction probabilities as a whole into account for confidence estimation.

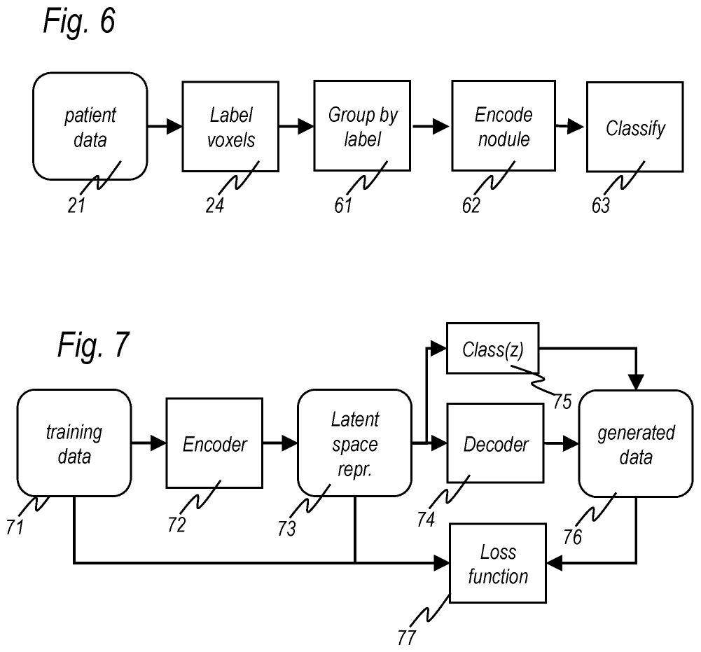

FIG. 6 schematically shows a further method of classifying nodules according to an embodiment of the disclosed subject matter. As an alternative to the histogram in action 26, the voxels forming a nodule group (action 61) can be passed to an encoder stage 62 of an encoder-decoder pair, in order to obtain a latent space representation of the nodule. The classification in action 63 can then be done based on the latent space representation instead of based on the histogram.

The grouping action 61 need not be very complicated in this case. For example, it can simply comprise selecting a block of 3D data around the centre of each detected nodule. For example, a block of 32×32×32 centred at a centre of the nodule may be provided to the encoder stage.

The encoder stage can be part of an encoder-decoder pair (EDP) as shown in FIG. 7. The encoder 72 is a neural network which takes data input x (e.g. nodule data 71) and outputs a latent space or representation space value z (latent space representation 73). The decoder 74 is also a neural network. It takes as input the latent space value z, and calculates an approximation of the input data x′. The loss function 77, used during training of the EDP, is designed to make the encoder and decoder work to minimize the difference between the actual and approximated inputs x and x′. A key aspect of the EDP is that the latent space z has a lower dimensionality than the input data. The latent space z is thus a bottleneck in the conversion of data x into x′, making it generally impossible to reproduce every detail of x exactly in x′. This bottleneck effectively forces the encoder/decoder pair to learn an ad-hoc compression algorithm that is suitable for the type of data x in the training set. Another way of looking at it, is that the encoder learns a mapping from the full space of x to a lower dimension manifold z that excludes the regions of the full space of x that contain (virtually) no data points.

The decoder 74 can be paired with a further function 75 that leans to determine a nodule classification from the latent space representation 73. During training, the classification is part of the generated data and accounted for in the loss function 77. The trained function 75 can be used in classification action 63 of FIG. 6.

An example EDP is an autoencoder. The most basic autoencoder has a loss function which, as a loss function, calculates an L1 or L2 norm of the generated data minus the training data. However, if the latent space is to have certain characteristics (such as smoothness), it is useful to also use aspects of the latent space as input in the loss function. For example, a variational autoencoder (Diederik P Kingma and Max Welling, “Auto-encoding variational Bayes”, Proceedings of the 2nd International Conference on Learning Representations, 2013) has a loss function that includes next to the standard reconstruction error an additional regularisation term (the KL divergence) in order to encourage the encoder to provide a better organisation of the latent space.

A feature of variational autoencoders is that, contrary to the most basic autoencoder, the latent space is stochastic. The latent variables are drawn from a prior p(z). The data x have a likelihood p(x|z) that is conditioned on the latent variables z. The encoder will learn a p(z|x) distribution.

In a further development of VAE's, a β parameter was introduced to add more weight to the KL divergence, in order to promote an even better organisation of the latent space, at the cost of some increase in the reconstruction error.

Autoencoders and VAE's are not the only possible EDP's that can be used. It is also possible to use a U-Net as an encoder-decoder. A U-Net EDP is similar to an EDP using a conventional Convolutional Neural Network encoder and decoder, with the difference that there are additional connections between encoder layers and the mirrored decoder layers, which bypass the latent space between the encoder and decoder. While it may seem counter-intuitive to have these latent space bypasses in order to promote a better latent space, these bypasses may actually help the encoder to reduce the reconstruction error without overburdening the latent space with storage of high-frequency image details which are important for the decoder to accurately recreate the input image (and thus to reduce the reconstruction error), but which are not important for the purposes of the latent space representation.

As a further refinement, the encoder may be built using a probabilistic U-Net. A probabilistic U-Net is able to learn a distribution over possible outcomes (such as segmentation) rather than a single most likely outcome/segmentation. Like VAEs, the probabilistic U-Nets use a stochastic variable distribution to draw latent space samples from. The probabilistic U-Net allows for hi-resolution encoding/decoding without much loss in the decoded images. It also allows the variability in the labelled image or other data (due to radiologist marking variability, measurement variability, etc) to be explicitly modelled.

Another way to improve the latent space representation is by including a Discriminator of a Generative Adversarial Network (GAN) in the loss function. The discriminator is separately trained to learn to distinguish the generated data from the original training data. The training process then involves training both the EDP and the loss function's discriminator. Usually, this is done by alternately training one and the other. Use of a GAN discriminator typically yields sharper and more realistic looking generated data than traditional reconstruction errors (e.g. L1 or L2 norm).

In FIG. 6, only the encoder 72 of the EDP is used in action 62. In action 63, a further model is used to determine a classification based on the latent space representation z output by the encoder in action 62. The model used in action 63 has been trained (as part of decoder 74 or separate function 75) to generate the correct classification based on manually labelled image data.

Combinations of specific features of various aspects of the disclosure may be made. An aspect of the disclosure may be further advantageously enhanced by adding a feature that was described in relation to another aspect of the disclosure.

It is to be understood that the disclosure is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

We claim:

1. A computer-implemented method for processing medical image data, the method comprising:
inputting, with one or more processors of one or more computation devices, medical image data into a model for nodule detection;
calculating, for at least one nodule detected by the model for nodule detection, a nodule histogram of all voxel intensities of said nodule; and
determining, from each nodule histogram, a nodule classification among a plurality of nodule classifications for at least one nodule.

2. The method of claim 1, wherein the nodule histogram is weighted with voxel prediction probabilities from the model for nodule detection.

3. The method of claim 1, wherein the model for nodule detection labels each voxel in the medical image data with a voxel label.

4. The method of claim 3, wherein the voxel label is selected from a label set comprising a nodule label and one or more non-nodule labels.

5. The method of claim 4, further comprising grouping voxels having a nodule label into nodule groups, wherein the histogram of all voxel intensities of a nodule is based on all voxels in a nodule group.

6. The method of claim 1, wherein the model for nodule detection uses a convolutional neural network (CNN).

7. The method of claim 1, further comprising calculating a maximum likelihood intensity in the nodule histogram, and wherein the classification is determined depending on the maximum likelihood intensity.

8. The method of claim 7, wherein an intensity range is divided in a plurality of intensity ranges, wherein each intensity range corresponds to a nodule classification among the plurality of nodule classifications.

9. The method of claim 1, wherein the plurality of nodule classifications comprises one or more of ground glass, part solid, solid, and calcified.

10. A computing system for processing medical image data, comprising:
one or more computation devices in a cloud computing environment and one or more storage devices accessible by the one or more computation devices, wherein the one or more computation devices comprise one or more processors, and wherein the one or more processors are programmed to:
input medical image data into a model for nodule detection;
calculate, for at least one nodule detected by the model for nodule detection, a nodule histogram of all voxel intensities of said nodule; and
determine, from each nodule histogram, a nodule classification among a plurality of nodule classifications for at least one nodule.

11. The system of claim 10, wherein the nodule histogram is weighted with voxel prediction probabilities from the model for nodule detection.

12. The system of claim 10, wherein the one or more processors are further programmed to label each voxel in the medical image data with a voxel label.

13. The system of claim 12, wherein the one or more processors are further programmed to select the voxel label from a label set comprising a nodule label and one or more non-nodule labels.

14. The system of claim 13, wherein the one or more processors are further programmed to group voxels having a nodule label into nodule groups, wherein the histogram of all voxel intensities of a nodule is based on all voxels in a nodule group.

15. The system of claim 10, wherein the model for nodule detection uses a convolutional neural network (CNN).

16. The system of claim 10, wherein the one or more processors are further programmed to calculate a maximum likelihood intensity in the nodule histogram, and wherein the classification is determined depending on the maximum likelihood intensity.

17. The system of claim 16, wherein an intensity range is divided in a plurality of intensity ranges, wherein each intensity range corresponds to a nodule classification among the plurality of nodule classifications.

18. The system of claim 10, wherein the plurality of nodule classifications comprises one or more of ground glass, part solid, solid, and calcified.
